(19) United States
(12) Patent Application Publication
(10) Pub. No.: US 2008/0070862 A1
(43) Pub. Date: Mar. 20, 2008

(54) Methods using glycosaminoglycans for the treatment of nephropathy

(76) Inventors: Morris Laster, Jerusalem (IL); Noa Shelach, Jerusalam (IL)

Correspondence Address: JONES DAY, 222 EAST 41ST ST, NEW YORK, NY, 10017, US

(21) Appl. No.: 11/903,486

(22) Filed: Sep. 21, 2007

Related U.S. Application Data
(63) Continuation of application No. 11/124,531, filed on May 6, 2005, now abandoned.
Application No. 11/124,531 is a continuation of application No. 10/170,063, filed on Jun. 12, 2002, now abandoned.
(60) Provisional application No. 60/298,132, filed on Jun. 12, 2001.

(51) Int. Cl. A61K 31/726; A61P 31/18

(52) U.S. Cl. 514/54

(57) ABSTRACT

The present invention relates to a method for the treatment of HIV-associated nephropathy by administration of glycosaminoglycans, and in particular, by the administration of sulodexide.

The present invention is a continuation of U.S. application Ser. No. 11/124,531 filed May 6, 2005, which is a continuation of U.S. application Ser. No. 10/170,063 filed Jun. 12, 2002, which in turn claims priority benefits of U.S. Provisional Application Ser. No. 60/298,132 filed Jun. 12, 2001, the disclosures of each of which are incorporated herein by reference in their entirety.

### 1. FIELD OF THE INVENTION

The present invention concerns methods for the treatment of renal diseases.

### 2. BACKGROUND OF THE INVENTION

Glycosaminoglycans, such as heparin, are routinely used in anticoagulant and antithrombotic therapies.

Sulodexide is a glycosaminoglycan (GAG) of natural origin extracted from mammalian intestinal mucosa and possesses an anticoagulant activity and a sulfation degree lower than that of heparin, as shown by Radhakrishnamurthy et al., 1978, Atherosclerosis 31:217-229. The preparation of Sulodexide is described in U.S. Pat. No. 3,936,351, which is incorporated herein by reference in its entirety.

Sulodexide is marketed in Europe under the trademark VESSEL DUE F® and is prescribed for the treatment of vascular pathologies with thrombotic risk such as peripheral occlusive arterial disease (POAD), healing of venous leg ulcers, and intermittent claudication (Harenberg, 1998, Med. Res. Rev. 18:1-20, Crepaldi et al., 1990, Atherosclerosis 81:233), cardiovasculopathies (Tramarin et al., 1987, Medical Praxis 8:1), cerebrovasculopathies (Sozzi, 1984, Eur. Rev. Med. Pharmacol. Sci. 6:295, and venous pathologies of the lower limbs (Cospite et al., 1992, Acta Therapeutica 18:149.

Kanway et al., 1985, Sem. Nephrol. 5:307 and Groggel et al., 1988, Kidney Int. 33:517 produced evidence of the probable role of glycosaminoglycans in helping the integrity and the functioning of the renal cells.

Canfield et al., 1978, Lab. Invest. 39:505 showed a decrease of membranal glycosaminoglycans in conditions of diabetic nephropathy. (Jensen, T., 1997, Pathogenesis of diabetic vascular disease: evidence for the role of reduced heparan sulfate proteoglycan. Diabetes 46 (Suppl. 2):S98-S100). This decrease may be mediated by decreased heparan sulfate production and/or sulfation (Raats, C. J. I., J. van den Born, and J. H. M. Berden, 2000, Glomerular heparan sulfate alterations: mechanisms and relevance for proteinuria. Kidney Int. 57:385-400).

U.S. Pat. No. 5,236,910 discloses the use of glycosaminoglycans for the treatment of diabetic nephropathy and diabetic neuropathy. U.S. Pat. No. 5,496,807 discloses a method of treatment of diabetic nephropathy by the administration of sulodexide.

Human immunodeficiency virus associated nephropathy (HIVAN) is an increasingly recognized complication of HIV infection. The disease occurs primarily in blacks. HIVAN has been described as an impending epidemic. It is estimated that at any given time, at least 10% of patients infected with the HIV virus will show evidence of HIVAN.

The initial sign of HIVAN is proteinuria. This can reach massive proportions with many patients being reported as having greater than 10 g of protein excreted in their urine per day. The proteinuria is followed by a rapid rise in serum creatinine. Typically, once the proteinuria becomes apparent, patients will progress from a normal serum creatinine (approximately 1 mg/dL) to renal failure within 6 months.

Histologically, the diagnosis of HIVAN is confirmed by the presence of either focal segmental or global glomerular sclerosis. There is also usually an interstitial infiltrate. Kidneys are typically large, about 13-15 cm in size, and are echogenic on renal ultrasound.

It is thought that HIVAN can be evident at any point in HIV disease, but most patients with HIVAN have CD4 counts of <200 cells/mL, which suggests that the HIVAN may be primarily a manifestation of a late stage of the HIV disease. The prognosis is poor, with end-stage renal failure typically occurring, in the absence of specific therapy, within weeks to months from the onset of the disease. For patients who subsequently require dialysis, mortality rate can approach 50% per year.

Treatment of HIVAN remains controversial. There have been several studies looking at the role of HAART, ACE Inhibitors, steroids and even cyclosporin in the treatment of HIVAN, with somewhat encouraging results. However, none of these studies is conclusive, as to date, there have been no randomized case-controlled trials. Most of the studies have been small and retrospective and many have included patients both with and without renal biopsy-proven HIVAN.

While diabetic nephropathy and HIVAN are both renal pathologies, there are marked differences between the two. Diabetic nephropathy is typically a slow evolving disease, the deterioration from the beginning of the nephrotic condition to final renal failure sometimes taking up to ten years. Against this the renal deterioration in HIVAN patients may be very rapid, with deterioration from onset of the disease to final renal failure lasting merely several weeks to several months.

Diabetic and HIV-associated nephropathies also differ in the protein and albumin secretion levels, typically HIVAN patients feature protein secretion rates which are about 3-5 times higher than those of diabetic nephropathy patients. The classic pathologic feature of HIVAN is the collapsing form of focal and segmental glomerulosclerosis, while diabetic nephropathy features a more wide-spread glomerulosclerosis, with thickening of the glomerular basement membranes, mesangial expansion and tubular and interstitial damage.

Another unique feature of HIVAN is the collapse and obliteration of capillary lumena.

One of the most distinctive features of HIVAN, is the presence of numerous tubuloreticular inclusions within the cytoplasm of glomerular and peritubular capillary endothelial cells.

Citation of a reference in this or any section of the specification shall not be construed as an admission that such reference is prior art to the present invention.

### 3. SUMMARY OF THE INVENTION

The present invention concerns a method of preventing, reducing or eliminating symptoms or complications of HIV-associated nephropathy, comprising: administering to a subject in need of such treatment an amount of glycosaminoglycans (GAGs), effective in inhibiting, reducing or eliminating one or more causes, symptoms or complications of HIV-associated nephropathy.

In a preferred embodiment, the glycosaminoglycan of the invention is sulodexide.

In an especially preferred embodiment of the invention the sulodexide is administered orally.

The present invention can be more fully explained by reference to the following detailed description and illustrative examples.

### 4. DETAILED DESCRIPTION OF THE INVENTION

The present invention encompasses methods for the prevention, reduction or elimination of symptoms or complications of HIV-associated nephropathy by administration to a patient, in need of such treatment, an effective amount of glycosaminoglycans.

Examples of glycosaminoglycans (GAG) are those acceptable in the therapeutic field such as: heparin and its pharmaceutically acceptable salts; low molecular weight heparins obtained by chemical or enzymatic depolymerization; chemically modified heparins, for instance through reactions of 0 and/or N sulfation or desulfation; dermatan sulfate and its low molecular weight fractions, hyaluronan, chondroitin sulfate, heparan sulfate, keratan sulfate and their low molecular weight fractions. The glycosaminoglycans may also comprise a combination or mixture of two or more of the above. Most preferably the GAG is sulodexide.

Sulodexide comprises about 80% iduronylglycosaminoglycan sulfate (IGGS), which is a fast-moving heparin fraction, and about 20% dermatan sulfate. The fast moving component, which is determined by its electrophoretic mobility in the barium-propanediamine system, is found in commercial heparin along with a slower moving component. IGGS has a low to medium molecular weight of about 7 kD and a lower anticoagulant activity than the slow moving heparin fraction and unfractionated heparin. Compared to heparin, IGGS has the same dimeric component but with lower amounts of iduronic acid-2-O-sulfate and a different amount of glucosamine N-acetylated-glucuronic acid dimer.

The term “sulodexide” in the context of the invention refers to a composition comprising from about 60% to about 90% iduronylglycosaminoglycan sulfate and between about 10% to about 40% dermatan sulfate. This term in the context of the present invention refers also to a pharmaceutically acceptable salt, solvate, hydrate, or clathrate of sulodexide.

The term “prevention, reduction or elimination of symptoms or complications of HIV-associated nephropathy” in the context of the present invention refers to: prevention of HIV-associated nephropathy before it occurs (for example if the treatment begins with the manifestation of initial clinical indications of HIV such as decrease in CD4-bearing cells), elimination of established HIVAN altogether (as determined, for example, by the return of renal functions parameters to normal), or reduction in the undesired symptoms of the disease manifested by the decrease in the severity of an existing condition of HIVAN. The reduction in the undesired symptoms may be determined for example by the improvement in renal function as compared to the function prior to treatment. Such remediation may be evident in a delay in the onset of renal failure (including dialysis or transplant) or in a decrease in the rate of the deterioration of renal functions as determined for example by the slowing of the rate of the increase of proteinuria or slowing the rate of the rise in serum creatinine or by the fall in the parameter of creatinine clearance or GFR), or decrease in at least one symptom or complication caused by HIVAN including hospitalization rate or mortality.

The method of administration according to the present invention, may be oral, mucosal, parenteral, intramuscular or transdermal. The dosage of the active ingredient will vary considerably depending on the mode of administration, the patient's age, weight and the patient's general condition, as well as the severity of the disease.

Where for example the administration is parenteral (intramuscular or transdermal) and the active ingredient is sulodexide, the dosage should be in the range of 25-400 mg/day, preferably 50-100 mg/day.

Preferably, the pharmaceutical composition is in the form of an oral preparation. Because of their ease of administration, tablets and capsules are preferred and represent the most advantageous oral dosage unit form wherein solid pharmaceutical excipients are employed. If desired, tablets may be coated by standard aqueous or non-aqueous techniques.

Preferably, the oral pharmaceutical composition used in the method of the invention may be administered in a single or divided dosage from to 1 to 4 times per day.

The pharmaceutical composition preferably comprises VESSEL DUE F® (Alfa Wassermann, Italy) which is a commercially available form of sulodexide. Preferred solid dosage forms of the pharmaceutical compositions are tablets or capsules which are coated or uncoated and the preferred dosage forms range from about 20 mg per day to about 1,000 mg per day, preferably from about 100 mg to about 400 mg per day, most preferably from about 200 to about 400 mg/day.

Oral Dosage Forms

Pharmaceutical compositions used in the method of the present invention suitable for oral administration may be presented as discrete pharmaceutical unit dosage forms, such as capsules, cachets, soft elastic gelatin capsules, tablets, caplets, or aerosol sprays, each containing a predetermined amount of the active ingredient, such as a powder or granules, or as a solution or a suspension in an aqueous liquid, a non-aqueous liquid, an oil-in-water emulsion, or a water-in-oil liquid emulsion. Dosage forms such as oil-in-water emulsions typically comprise surfactants such as anionic phosphate ester or lauryl sulfates, but other types of surfactants such as cationic or nonionic surfactants may be used in the compositions of the present invention. See, generally, Remington's Pharmaceutical Sciences, 18^{th} ed., Mack Publishing, Easton Pa. (1990).

Pharmaceutical compositions of the present invention suitable for oral administration may be formulated as a pharmaceutical composition in a soft elastic gelatin capsule unit dosage form by using conventional methods well known in the art. See, e.g., Ebert, 1977, Pharm. Tech. 1(5):44-50. Pharmaceutical compositions in the form of capsules or tablets coated by an enterosoluble gastro resistant film and which contains a lyophilisate consisting of glycosaminoglycan, a thickening agent, and a surfactant have been previously described in U.S. Pat. No. 5,252,339, which is incorporated herein by reference in its entirety.

Soft elastic gelatin capsules have a soft, globular gelatin shell somewhat thicker than that of hard gelatin capsules, wherein a gelatin is plasticized by the addition of plasticizing agent, e.g., glycerin, sorbitol, or a similar polyol. Varying the type of gelatin used and the amounts of plasticizer and water may change the hardness of the capsule shell. The soft gelatin shells may contain a preservative, such as methyl and propylparabens and sorbic acid, to prevent the growth of fungi. The active ingredient may be dissolved or suspended in a liquid vehicle or carrier, such as vegetable or mineral oils, glycols, such as polyethylene glycol and propylene glycol, triglycerides, surfactants, such as polysorbates, or a combination thereof.

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, Excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL® PH-101, AVICEL® PH-103 AVICEL® RC-581, AVICEL® PH-105 (available from FMC Corporation, American Viscose Division, Avicel Sales, Marcus Hook, PA), and mixtures thereof. A specific binder is a mixture of microcrystalline cellulose and sodium carboxymethyl cellulose sold as AVICEL® RC-581. Suitable anhydrous or low moisture excipients or additives include AVICEL® PH-103 and Starch 1500 LM.

Examples of fillers suitable for use in the pharmaceutical compositions and dosage forms disclosed herein include, but are not limited to talc, calcium carbonate (e.g. granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Pharmaceutical stabilizers may also be used to stabilize the compositions of the invention. Acceptable stabilizers include but are not limited to L-cysteine hydrochloride, glycine hydrochloride, malic acid, sodium metabisulfite, citric acid, tartaric acid and L-cysteine dihydrochloride.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

In addition to the common dosage forms set out above, the compounds of the present invention may also be administered by controlled release means or delivery devices that are well known to those of ordinary skill in the art, such as those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566.

These pharmaceutical compositions can be used to provide slow or controlled-release of one or more of the active ingredients therein using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable controlled-release formulations known to those of ordinary skill in the art, including those described herein, may be readily selected for use with the pharmaceutical compositions of the invention. Thus, single unit dosage forms suitable for oral administration, such as tablets, capsules, gelcaps, caplets, and the like, that are adapted for controlled-release are encompassed by the present invention.

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

Co-Administration

The method of treatment of the present invention may also include co-administration of other therapeutically effective agents, together with the administration of the GAG, preferably together with the administration of the sulodexide. Examples of such agents that can be co-administered with the active ingredients (GAGs and preferably sulodexide) of the method of the present invention are: cyclosporin, glucocorticoids, anti-HIV medicaments (such as AZT alone or in combination with ddI), ACE inhibitors, A2 blockers, HAART (3TC, d4T, nelfinavir or others), anti-TGF-β agents, pain relievers, antibiotics (including antibacterials, antituberculosis, antifungals, antivirals, antiparasitic agents and others), anti-cancer chemotherapeutics as well as any other medicament used to treat HIV patients.

Assessment of Renal Function

In order to assess the efficacy of the method of the invention, serial measurements of renal function of the patients must be determined. Quantitative assessment of renal function, and parameters of renal dysfunction are well known in the art and can be found, for example, in Levey, 1993, Assessing the effectiveness of therapy to prevent the progression of renal disease, Am J Kidney Dis. 22(1):207-214.

Examples of assays for the determination of renal function/dysfunction are:

Serum creatinine level;

Creatinine clearance rate;

24-hour urinary protein secretion;

Glomerular filtration rate (GFR);

Urinary albumin creatinine ratio (ACR);

Albumin excretion rate (AER); and

Renal biopsy.

The following series of examples are presented by way of illustration and not by way of limitation on the scope of the invention.

### 5. EXAMPLES

### Example 1

### Treatment of HIVAN by Administration of Sulodexide

75 HIV patients (documented by positive HIV serology) and featuring HIVAN (as determined by glomerulosclerosis found by renal biopsy) are studied. The patients included in the study have a serum creatinine between 1.5 mg/dL to 3.5 mg dL and proteinura greater than 2 g/24 hours.

The patients are randomly divided into 3 groups: one administered with placebo (morning and evening); the second with 200 mg sulodexide a day (sulodexide morning and placebo evening); and the third administered with 400 mg sulodexide a day (200 mg morning and 200 mg night).

Treatment period is 24 weeks.

Patients return to the clinic every 4 weeks. During each visit the following parameters are monitored:

1) Adverse events monitoring;

2) Concomitant medications assessment;

3) Study medication compliance check (i.e., patients will be queried about their level of compliance with taking their study medication, and the number of remaining gel caps will be counted);

4) Routine physical examination including vital signs, and weight;

5) Blood samples for measuring renal profile, hepatic profile, bone profile, CBC, PT, and PTT; and

6) Urine sample for measuring Protein/Creatiine Ratio (PCR).

At visit 1 and visit 8, creatinine clearance and serum TGF-β protein levels are measured.

4 weeks after termination of the treatment patients undergo final evaluation wherein the following parameters are monitored:

1. Concomitant medications taken during the preceding month;

2. Adverse events monitoring;

3. Physical examination including weight and vital signs;

4. Blood samples for measuring renal profile, hepatic profile, bone profile CBC, PT and PTT;

5. Urine sample for measuring PCR;

6. EKG; and

7. Chest x-ray.

The primary efficacy endpoints are the rates of change of serum creatinine and urinary PCR (protein/creatinine ratio), between baseline and after 24 weeks of therapy, comparing the two dosage treatment groups to each other and to the placebo treated patients.

The secondary efficacy endpoints are the rates of treatment failure (defined as patients requiring initiation of corticosteroid as a result of doubling of serum creatinine), of renal failure (defined by serum creatinine greater than 6 mg/dL, initiation of dialysis, renal transplantation or death from renal causes (azotemia, hyperkalemia or pulmonary edema of non cardiac origin)), rate and time to azotemic death, creatinine clearance, rates of hospitalization and mortality rates, comparing the sulodexide treatment groups to each other and to placebo treated patients.

The data is analyzed using analysis of covariance (ANCOVA). A last observation carried forward technique will be utilized to handle missing data including cases of documented patient death. Secondary endpoints are analyzed using a chi square analysis with a Yates correction, ANCOVA, and a log rank test where appropriate.

Results:

The following are the results for serum creatinine of the first two patients enrolled as determined in visit 1 (prior to treatment, treatment was started on visit 2 weeks after visit 1), visit 3 (after 4 weeks of treatment and 6 weeks from visit) and visit 4 (after 8 weeks of treatment and 10 weeks after visit 1).

Patient 1 (subject No. 101) serum creatinine (mg/dL)

Visit 1: 2.06

Visit 3: 2.42

Visit 4: 2.80

Change between visit 3 and beginning of treatment: 0.36

Change between visit 4 and beginning of treatment: 0.74

Patient 2 (subject No. 201) serum creatinine (mg/dL)

Visit 1: 3.06

Visit 3: 2.60

Visit 4: 2.65

Change between visit 3 and beginning of treatment: −0.46

Change between visit 4 and beginning of treatment: −0.41

### Example 2

### Transgenic Mice Model

20 transgenic mice, that develop renal disease similar to HIVAN, are used according to the teaching of Bird et al., 1998, J. Am. Soc. Naphrol. 9(8):1441-1447. Wild type mice are used as a control for healthy individuals.

Wild type or transgenic mice are each divided into two groups: treatment and control. Treatment groups are administered with sulodexide administered in the drinking water in an amount of 3 mg/kg/ for a period of 100 days. Non-treated transgenic or wild type mice were not administered with sulodexide but otherwise kept under the same conditions.

Serum creatinine, urinary protein excretion and plasma concentration of TGF-p are compared among the different groups. Kidney biopsies are also performed on all mice at the end of the 100-day study.

The results are compared for wild type treated and untreated mice, as well as renally diseased, treated and untreated, transgenic mice.

The study is repeated for very young transgenic mice before the manifestations of renal dysfunction in order to determine the efficacy of sulodexide in the prevention of the renal disease.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

What is claimed is:

1. A method of preventing, reducing or eliminating symptoms or complications of HIV-associated nephropathy (HIVAN) in a patient comprising: administering to a patient, in need of such treatment, an amount of glycosaminoglycans effective in preventing, reducing or eliminating one or more causes, symptoms or complications of HIVAN.

2. A method according to claim 1 wherein the glycosaminoglycan is selected from the group consisting of heparin and its pharmaceutically acceptable salts; low molecular weight heparins obtained by chemical or enzymatic depolymerization; chemically modified heparins; dermatan sulfate and its low molecular weight fractions; hyaluronan, chondroitin sulfate, heparan sulfate, keratan sulfate and their low molecular weight fractions, and a combination or mixture of two or more of the above.

3. A method according to claim 1, wherein the glycosaminoglycan is sulodexide, or a pharmaceutically acceptable salt, solvate, hydrate or clathrate of sulodexide.

4. A method according to claim 3, wherein the sulodexide is administered parenterally.

5. A method according to claim 4, wherein the sulodexide is parenterally administered in ranges from about 25 mg/day to about 400 mg/day.

6. A method according to claim 3, wherein the sulodexide is administered orally.

7. A method according to claim 6, wherein the sulodexide is administered orally, and ranges from about 20 mg/day to about 1,000 mg/day.

8. A method according to claim 7, wherein the sulodexide is administered orally and ranges in mount from about 100 mg/day to about 400 mg/day.
